(19) United States
(12) Patent Application Publication
(10) Pub. No.: US 2002/0156471 A1
(43) Pub. Date: Oct. 24, 2002

(54) Method for treatment of tissue

(76) Inventors: Roger A. Stern, Cupertino, CA (US); Mitchell Levinson, Pleasanton, CA (US); Bryan Weber, Livermore, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/117,990

(22) Filed: Apr. 5, 2002

Related U.S. Application Data
(63) Continuation-in-part of application No. 10/072,475, filed on Feb. 6, 2002.
(63) Continuation-in-part of application No. 10/072,610, filed on Feb. 6, 2002.
Application No. 10/072,610 is a continuation-in-part of application No. 09/522,275, filed on Mar. 9, 2000, now Pat. No. 6,413,255.
(60) Provisional application No. 60/123,440, filed on Mar. 9, 1999.

(51) Int. Cl. A61B 18/18; A61N 1/04

(52) U.S. Cl. 606/41; 607/101; 607/102; 607/145

(57) ABSTRACT

A method for creating a tissue effect provides a substrate with a releasable coating. At least a portion of the releasable coating is released on a selected skin epidermis surface to create a marked skin epidermis surface. The marked skin epidermis surface is used to provide a guide for delivery of energy from an energy source to a tissue site through at least a portion of the marked skin epidermis surface.

### CROSS-REFERENCE TO RELATED APPLICATIONS

This application is a continuation-in-part of U.S. Ser. No. 10/072,475 filed Feb. 6, 2002 and a continuation-in-part of U.S. Ser. No. 10/072,610 filed Feb. 6, 2002, both of which are continuations-in-part of U.S. Ser. No. 09/522,275, filed Mar. 9, 2000, which claims the benefit of U.S. Ser. No. 60/123,440, filed Mar. 9, 1999, all fully incorporated herein by reference.

### FIELD OF THE INVENTION

This invention relates generally to methods and apparatus that deliver energy through a skin surface to create a desired tissue effect, and more particularly to methods and apparatus that provide a marking of a skin surface followed by delivery of energy through at least a portion of the skin surface to create a desired tissue effect.

### DESCRIPTION OF RELATED ART

The human skin is composed of two elements: the epidermis and the underlying dermis. The epidermis with the stratum corneum serves as a biological barrier to the environment. In the basilar layer of the epidermis, pigment-forming cells called melanocytes are present. They are the main determinants of skin color.

The underlying dermis provides the main structural support of the skin. It is composed mainly of an extra-cellular protein called collagen. Collagen is produced by fibroblasts and synthesized as a triple helix with three polypeptide chains that are connected with heat labile and heat stable chemical bonds. When collagen-containing tissue is heated, alterations in the physical properties of this protein matrix occur at a characteristic temperature. The structural transition of collagen contraction occurs at a specific “shrinkage” temperature. The shrinkage and remodeling of the collagen matrix with heat is the basis for the technology.

Collagen crosslinks are either intramolecular (covalent or hydrogen bond) or intermolecular (covalent or ionic bonds). The thermal cleavage of intramolecular hydrogen crosslinks is a scalar process that is created by the balance between cleavage events and relaxation events (reforming of hydrogen bonds). No external force is required for this process to occur. As a result, intermolecular stress is created by the thermal cleavage of intramolecular hydrogen bonds. Essentially, the contraction of the tertiary structure of the molecule creates the initial intermolecular vector of contraction.

Collagen fibrils in a matrix exhibit a variety of spatial orientations. The matrix is lengthened if the sum of all vectors acts to distract the fibril. Contraction of the matrix is facilitated if the sum of all extrinsic vectors acts to shorten the fibril. Thermal disruption of intramolecular hydrogen bonds and mechanical cleavage of intermolecular crosslinks is also affected by relaxation events that restore preexisting configurations. However, a permanent change of molecular length will occur if crosslinks are reformed after lengthening or contraction of the collagen fibril. The continuous application of an external mechanical force will increase the probability of crosslinks forming after lengthening or contraction of the fibril.

Hydrogen bond cleavage is a quantum mechanical event that requires a threshold of energy. The amount of (intramolecular) hydrogen bond cleavage required corresponds to the combined ionic and covalent intermolecular bond strengths within the collagen fibril. Until this threshold is reached, little or no change in the quaternary structure of the collagen fibril will occur. When the intermolecular stress is adequate, cleavage of the ionic and covalent bonds will occur. Typically, the intermolecular cleavage of ionic and covalent bonds will occur with a ratcheting effect from the realignment of polar and nonpolar regions in the lengthened or contracted fibril.

Cleavage of collagen bonds also occurs at lower temperatures but at a lower rate. Low-level thermal cleavage is frequently associated with relaxation phenomena in which bonds are reformed without a net change in molecular length. An external force that mechanically cleaves the fibril will reduce the probability of relaxation phenomena and provides a means to lengthen or contract the collagen matrix at lower temperatures while reducing the potential of surface ablation.

Soft tissue remodeling is a biophysical phenomenon that occurs at cellular and molecular levels. Molecular contraction or partial denaturization of collagen involves the application of an energy source, which destabilizes the longitudinal axis of the molecule by cleaving the heat labile bonds of the triple helix. As a result, stress is created to break the intermolecular bonds of the matrix. This is essentially an immediate extra-cellular process, whereas cellular contraction requires a lag period for the migration and multiplication of fibroblasts into the wound as provided by the wound healing sequence. In higher developed animal species, the wound healing response to injury involves an initial inflammatory process that subsequently leads to the deposition of scar tissue.

The initial inflammatory response consists of the infiltration by white blood cells or leukocytes that dispose of cellular debris. Seventy-two hours later, proliferation of fibroblasts at the injured site occurs. These cells differentiate into contractile myofibroblasts, which are the source of cellular soft tissue contraction. Following cellular contraction, collagen is laid down as a static supporting matrix in the tightened soft tissue structure. The deposition and subsequent remodeling of this nascent scar matrix provides the means to alter the consistency and geometry of soft tissue for aesthetic purposes.

In light of the preceding discussion, there are a number of dermatological procedures that lend themselves to treatments which deliver thermal energy to the skin and underlying tissue to cause a contraction of collagen, and/or initiate a would healing response. Such procedures include skin remodeling/resurfacing, wrinkle removal, and treatment of the sebaceous glands, hair follicles adipose tissue and spider veins.

Currently available technologies that deliver thermal energy to the skin and underlying tissue include Radio Frequency (RF), optical (laser) and other forms of electromagnetic energy. However, these technologies have a number of technical limitations and clinical issues which limit the effectiveness of the treatment and/or preclude treatment altogether. These issues include the following: i) achieving a uniform thermal effect across a large area of tissue, ii) controlling the depth of the thermal effect to target selected tissue and prevent unwanted thermal damage to both target and non-target tissue, iii) reducing adverse tissue effects such as bums, redness blistering, iv) replacing the practice of delivery energy/treatment in a patchwork fashion with a more continuous delivery of treatment (e.g. by a sliding or painting motion), v) improving access to difficult-to-reach areas of the skin surface and vi) reducing procedure time and number of patient visits required to complete treatment. As will be discussed herein the current invention provides an apparatus for solving these and other limitations.

One of the key shortcomings of currently available RF technology for treating the skin is the edge effect phenomenon. In general, when RF energy is being applied or delivered to tissue through an electrode which is in contact with that tissue, the current patterns concentrate around the edges of the electrode, sharp edges in particular. This effect is generally known as the edge effect. In the case of a circular disc electrode, the effect manifests as a higher current density around the perimeter of that circular disc and a relatively low current density in the center. For a square-shaped electrode there is typically a high current density around the entire perimeter, and an even higher current density at the corners where there is a sharp edge.

Edge effects cause problems in treating the skin for several reasons. First, they result in a non-uniform thermal effect over the electrode surface. In various treatments of the skin, it is important to have a uniform thermal effect over a relatively large surface area, particularly for dermatologic treatments. Large in this case being on the order of several square millimeters or even several square centimeters. In electrosurgical applications for cutting tissue, there typically is a point type applicator designed with the goal of getting a hot spot at that point for cutting or even coagulating tissue. However, this point design is undesirable for creating a reasonably gentle thermal effect over a large surface area. What is needed is an electrode design to deliver uniform thermal energy to skin and underlying tissue without hot spots.

A uniform thermal effect is particularly important when cooling is combined with heating in skin/tissue treatment procedure. As is discussed below, a non-uniform thermal pattern makes cooling of the skin difficult and hence the resulting treatment process as well. When heating the skin with RF energy, the tissue at the electrode surface tends to be warmest with a decrease in temperature moving deeper into the tissue. One approach to overcome this thermal gradient and create a thermal effect at a set distance away from the electrode is to cool the layers of skin that are in contact with the electrode. However, cooling of the skin is made difficult if there is a non-uniform heating pattern. If the skin is sufficiently cooled such that there are no burns at the corners of a square or rectangular electrode, or at the perimeter of a circular disc electrode, then there will probably be overcooling in the center and there won't be any significant thermal effect (i.e. tissue heating) under the center of the electrode.

If the cooling effect is decreased to the point where there is a good thermal effect in the center of the electrode, then there probably will not be sufficient cooling to protect tissue in contact with the edges of the electrode. As a result of these limitations, in the typical application of a standard electrode there is usually an area of non-uniform treatment and/or burns on the skin surface. So uniformity of the heating pattern is very important. It is particularly important in applications treating skin where collagen-containing layers are heated to produce a collagen contraction response for tightening of the skin. For this and related applications, if the collagen contraction and resulting skin tightening effect are non-uniform, then a medically undesirable result may occur.

There is a need for an improved methods and systems for delivering energy to selected tissue sites through the skin with minimal damage of the skin surface. There is another need for methods and systems that provide marking of a skin surface and the delivery of energy to a selected tissue site to achieve a desired tissue effect. There is a further need for methods and systems that provide marking of a skin surface and the delivery of energy to a selected tissue site to achieve a desired therapeutic effect at the skin surface. There is a yet another need for methods and systems that provide marking of a skin surface and the delivery of energy to a selected tissue site to achieve selected scar collagen formation.

### SUMMARY OF THE INVENTION

Accordingly, an object of the invention is to provide devices and methods that mark a skin surface, deliver energy through the skin surface and achieve a desired tissue effect.

Another object of the invention is to provide devices and methods that mark a skin surface, deliver energy through the skin surface and achieved a desired therapeutic effect at the skin surface.

Yet another object of the invention is to provide devices and methods that mark a skin surface, deliver energy through the skin surface and create scar collagen at a selected tissue site.

These and other objects of the present invention are achieved in a method for creating a desired tissue effect. A skin surface is marked to create a marked skin surface. A handpiece is provided that includes a handpiece assembly coupled to an electrode assembly with at least one RF electrode that is capacitively coupled to a skin surface when at least a portion of the RF electrode is in contact with the skin surface. RF energy is delivered from the RF electrode assembly to at least a portion of the marked skin surface.

In another embodiment of the present invention, a method for creating a tissue effect marks a skin epidermis surface. An energy source is provided and a reverse thermal gradient is created through at least a portion of the skin epidermis surface. The reverse thermal gradient occurs when a temperature of the skin epidermis surface is lower than an underlying collagen containing tissue site. Energy is delivered from the energy source through the skin epidermis surface to the collagen containing tissue site for a sufficient time to induce collagen formation in the collagen containing tissue site while minimizing cellular necrosis of the skin epidermis surface to create a desired tissue effect.

In another embodiment of the present invention, a method for creating a desired tissue effect marks a skin epidermis surface to create a marked skin epidermis surface. An energy source is provided. At least a portion of the marked skin epidermis surface is cooled. Thermal energy is delivered to tissue underlying the at least a portion of the marked skin epidermis surface without creating substantial necrosis at the skin epidermis surface. A desired tissue effect is created.

In another embodiment of the present invention, a method for creating a desired tissue effect marks a skin epidermis surface to create a marked skin epidermis surface. An energy delivery surface of an electromagnetic delivery device is positioned on at least a portion of the marked skin epidermis surface. A reverse thermal gradient is created on at least a portion of the marked skin epidermis surface. The reverse thermal gradient cools the skin epidermis surface while heating underlying tissue. A temperature of the marked epidermis skin surface is lower than a temperature of the underlying tissue. At least a portion of the underlying tissue is contracted while cellular destruction of the marked skin epidermis surface is minimized. A desired tissue effect is created.

In another embodiment of the present invention, a method for creating a tissue effect provides a substrate with a releasable colorant. The substrate is applied to a selected skin epidermis surface to mark a skin surface with the colorant. An energy source is provided. A reverse thermal gradient is created through at least a portion of the skin epidermis surface where a temperature of the skin epidermis surface is lower than an underlying collagen containing tissue site. Energy is delivered from the energy source through the skin epidermis surface to the collagen containing tissue site for a sufficient time to induce collagen formation in the collagen containing tissue site while minimizing cellular necrosis of the skin epidermis surface. A desired tissue effect is created.

In another embodiment of the present invention, a kit is provided that includes a substrate with a releasable colorant coating. Also included is a handpiece that with a handpiece assembly coupled to an electrode assembly with at least one RF electrode that is capacitively coupled to a skin surface when at least a portion of the RF electrode is in contact with the skin surface.

In another embodiment of the present invention, a method for creating a tissue effect provides a substrate with a releasable coating. At least a portion of the releasable coating is released on a selected skin epidermis surface to create a marked skin epidermis surface. The marked skin epidermis surface is used to provide a guide for delivery of energy from an energy source to a tissue site through at least a portion of the marked skin epidermis surface. This energy source may be from a variety of methods including but not limited to lasers, radio-frequency electricity, microwave, ultrasound or heat.

### BRIEF DESCRIPTION OF THE DRAWINGS

FIG. 1 is a cross-sectional view of one embodiment of the handpiece of the present invention.

FIG. 2 is an exploded view of the FIG. 1 insert assembly.

FIG. 3 is a close-up view of one embodiment of an RF electrode of the present invention.

FIG. 4 is another cross-sectional view of a portion of the handpiece housing from FIG. 1.

FIG. 5 is a cross-sectional view of the insert from FIG. 1.

FIG. 6 is a cross-sectional view of one embodiment of marking device and a substrate with a releasable colorant coating that is used in one embodiment of the present invention.

FIG. 7 is a cross-sectional view of another embodiment of a substrate with a releasable colorant coating that is used in a method of the present invention.

FIG. 8 is a cross-sectional view of another embodiment of a substrate with a releasable colorant coating that is used in a method of the present invention.

### DETAILED DESCRIPTION

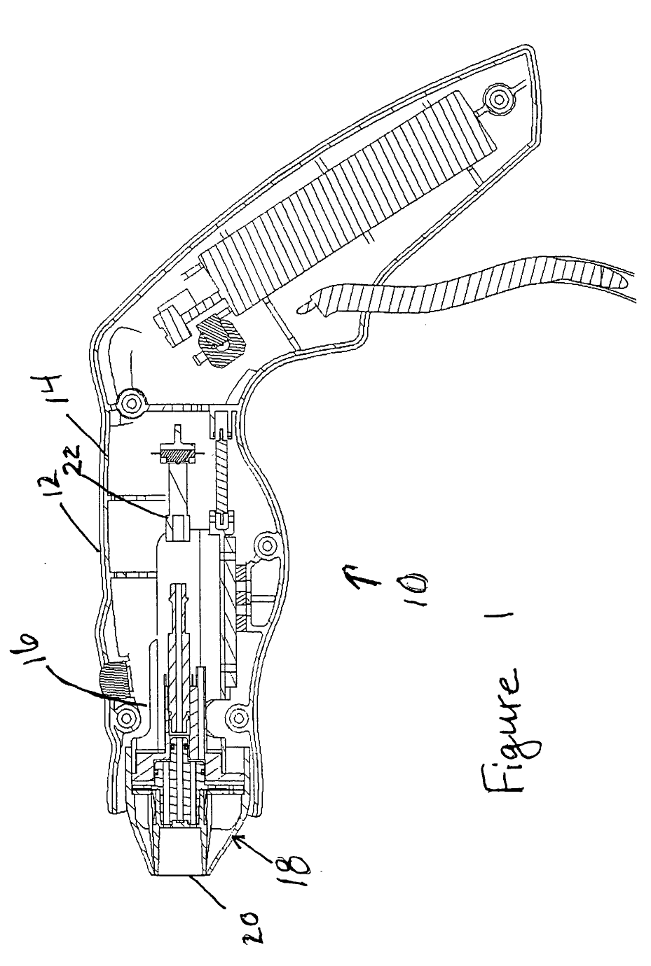

Referring now to FIG. 1, one embodiment of the present invention is a handpiece 10 with a handpiece assembly 12. Handpiece assembly 12 includes a handpiece housing 14 and a cooling fluidic medium valve member 16. An electrode assembly 18 is coupled to handpiece housing 14. Electrode assembly 18 has a least one RF electrode 20 that is capacitively coupled to a skin surface when at least a portion of RF electrode 20 is in contact with the skin surface. Without limiting the scope of the present invention, RF electrode 20 can have a thickness in the range of 0.010 to 1.0 mm.

Handpiece 10 provides a more uniform thermal effect in tissue at a selected depth, while preventing or minimizing thermal damage to the skin surface and other non-target tissue. Handpiece 10 is coupled to an RF generator. RF electrode 20 can be operated either in mono-polar or bi-polar modes. Handpiece 10 is configured to reduce, or preferably eliminate edge effects and hot spots. The result is an improved aesthetic result/clinical outcome with an elimination/reduction in adverse effects and healing time.

A fluid delivery member 22 is coupled to cooling fluidic medium valve member 16. Fluid delivery member 22 and cooling fluidic medium valve member 16 collectively form a cooling fluidic medium dispensing assembly. Fluid delivery member 16 is configured to provide an atomizing delivery of a cooling fluidic medium to RF electrode 20. The atomizing delivery is a mist or fine spray. A phase transition, from liquid to gas, of the cooling fluidic medium occurs when it hits the surface of RF electrode 20. The transition from liquid to gas creates the cooling. If the transition before the cooling fluidic medium hits RF electrode 20 the cooling of RF electrode 20 will not be as effective.

In one embodiment, the cooling fluidic medium is a cryogenic spray, commercially available from Honeywell, Morristown, N.J. A specific example of a suitable cryogenic spray is R134A₂, available from Refron, Inc., 38-18 33^{rd} St, Long Island City, N.Y. 11101. The use of a cryogenic cooling fluidic medium provides the capability to use a number of different types of algorithms for skin treatment. For example, the cryogenic cooling fluidic medium can be applied milliseconds before and after the delivery of RF energy to the desired tissue. This is achieved with the use of cooling fluidic medium valve member 16 coupled to a cryogen supply, including but not limited to a compressed gas canister. In various embodiments, cooling fluidic medium valve member 16 can be coupled to a computer control system and/or manually controlled by the physician by means of a foot switch or similar device.

A key advantage of providing a spray, or atomization, of cryogenic cooling fluidic medium is the availability to implement rapid on and off control. Cryogenic cooling fluidic medium allows more precise temporal control of the cooling process. This is because cooling only occurs when the refrigerant is sprayed and is in an evaporative state, the latter being a very fast short-lived event. Thus, cooling ceases rapidly after the cryogenic cooling fluidic medium is stopped. The overall effect is to confer very precise time on-off control of cryogenic cooling fluidic medium.

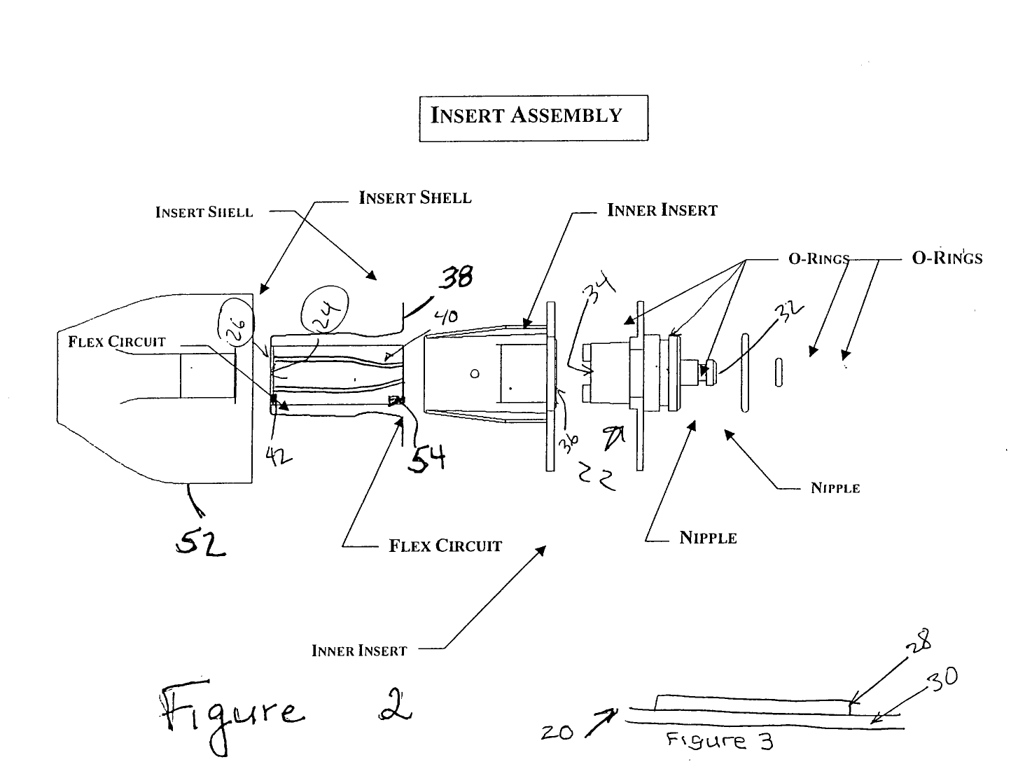

Referring now to FIG. 2, fluid delivery member 22 can be positioned in handpiece housing 14 or electrode assembly 18. Fluid delivery member 22 is configured to controllably deliver a cooling fluidic medium to a back surface 24 of RF electrode 20 and maintain back surface 24 at a desired temperature. The cooling fluidic medium evaporatively cools RF electrode 20 and maintains a substantially uniform temperature of front surface 26 of RF electrode 20. Front surface 26 can be sufficiently flexible and conformable to the skin, but still have sufficient strength and/or structure to provide good thermal coupling when pressed against the skin surface.

RF electrode 20 then conductively cools a skin surface that is adjacent to a front surface 26 of RF electrode 20. Suitable fluidic media include a variety of refrigerants such as R134A and freon. Fluid delivery member 22 is configured to controllably deliver the cooling fluidic medium to back surface 24 at substantially any orientation of front surface 26 relative to a direction of gravity. A geometry and positioning of fluid delivery member 22 are selected to provide a substantially uniform distribution of cooling fluidic medium on back surface 24. The delivery of the cooling fluidic medium can be by spray of droplets or fine mist, flooding back surface 24, and the like. Cooling occurs at the interface of the cooling fluidic medium with atmosphere, which is where evaporation occurs. If there is a thick layer of fluid on back surface 24 the heat removed from the treated skin will need to pass through the thick layer of cooling fluidic medium, increasing thermal resistance. To maximize cooling rates, it is desirable to apply a very thin layer of cooling fluidic medium. If RF electrode 20 is not horizontal, and if there is a thick layer of cooling fluidic medium, or if there are large drops of cooling fluidic medium on back surface 24, the cooling fluidic medium can run down the surface of RF electrode 20 and pool at one edge or corner, causing uneven cooling. Therefore, it is desirable to apply a thin layer of cooling fluidic medium with a fine spray.

In various embodiments, RF electrode 20, as illustrated in FIG. 3, has a conductive portion 28 and a dielectric portion 30. Conductive portion 28 can be a metal including but not limited to copper, gold, silver, aluminum and the like. Dielectric portion 30 can be made of a variety of different materials including but not limited to polyimide, and the like. Other dielectric materials include but are not limited to silicon, sapphire, diamond, zirconium-toughened alumina (ZTA), alumina and the like. Dielectric portion 30 can be positioned around at least a portion, or the entirety of a periphery of conductive portion 28. Suitable materials for a dielectric portion 30 include, but are not limited to, Teflon® and the like, silicon nitride, polysilanes, polysilazanes, polyimides, Kapton and other polymers, antenna dielectrics and other dielectric materials well known in the art. In another embodiment, RF electrode 20 is made of a composite material, including but not limited to gold-plated copper, copper-polyimide, silicon/silicon-nitride and the like.

Dielectric portion 30 creates an increased impedance to the flow of electrical current through RF electrode 20. This increased impedance causes current to travel a path straight down through conductive portion 28 to the skin surface. Electric field edge effects, caused by a concentration of current flowing out of the edges of RF electrode 20, are reduced.

Dielectric portion 30 produces a more uniform impedance through RF electrode 20 and causes a more uniform current to flow through conductive portion 28. The resulting effect minimizes or even eliminates, edge effects around the edges of RF electrode 20.

In one embodiment, conductive portion 28 adheres to dielectric portion 30 which can be substrate with a thickness, by way of example and without limitation, of about 0.001″. This embodiment is similar to a standard flex circuit board material commercially available in the electronics industry. In this embodiment, dielectric portion 30 is in contact with the tissue, the skin, and conductive portion 28 is separated from the skin. The thickness of the dielectric portion 30 can be decreased by growing conductive portion 28 on dielectric portion 30 using a variety of techniques, including but not limited to, sputtering, electro deposition, chemical vapor deposition, plasma deposition and other deposition techniques known in the art. Additionally, these same processes can be used to deposit dielectric portion 30 onto conductive portion 28. In one embodiment dielectric portion 30 is an oxide layer which can be grown on conductive portion 28. An oxide layer has a low thermal resistance and improves the cooling efficiency of the skin compared with many other dielectrics such as polymers.

Fluid delivery member 22 has an inlet 32 and an outlet 34. Outlet 34 can have a smaller cross-sectional area than a cross-sectional area of inlet 32. In one embodiment, fluid delivery member 22 is a nozzle 36.

Cooling fluidic medium valve member 16 can be configured to provide a pulsed delivery of the cooling fluidic medium. Pulsing the delivery of cooling fluidic medium is a simple way to control the rate of cooling fluidic medium application. In one embodiment, cooling fluidic medium valve member 16 is a solenoid valve. An example of a suitable solenoid valve is a solenoid pinch valve manufactured by the N-Research Corporation, West Caldwell, N.J. If the fluid is pressurized, then opening of the valve results in fluid flow. If the fluid is maintained at a constant pressure, then the flow rate is constant and a simple open/close solenoid valve can be used, the effective flow rate being determined by the pulse duty cycle. A higher duty cycle, close to 100% increases cooling, while a lower duty cycle, closer to 0%, reduces cooling. The duty cycle can be achieved by turning on the valve for a short duration of time at a set frequency. The duration of the open time can be 1 to 50 milliseconds or longer. The frequency of pulsing can be 1 to 50 Hz or faster.

Alternatively, cooling fluidic medium flow rate can be controlled by a metering valve or controllable-rate pump such as a peristaltic pump. One advantage of pulsing is that it is easy to control using simple electronics and control algorithms.

Electrode assembly 18 is sufficiently sealed so that the cooling fluidic medium does not leak from back surface 24 onto a skin surface in contact with a front surface of RF electrode 20. This helps provide an even energy delivery through the skin surface. In one embodiment, electrode assembly 18, and more specifically RF electrode 20, has a geometry that creates a reservoir at back surface 24 to hold and gather cooling fluidic medium that has collected at back surface 24. Back surface 24 can be formed with “hospital corners” to create this reservoir. Optionally, electrode assembly 18 includes a vent 38 that permits vaporized cooling fluidic medium to escape from electrode assembly 18. This reduces the chance of cooling fluidic medium collecting at back surface 24. This can occur when cooling fluidic medium is delivered to back surface 24 in vapor form and then, following cooling of back surface 24, the vapor condenses to a liquid.

Vent 38 prevents pressure from building up in electrode assembly 18. Vent 38 can be a pressure relief valve that is vented to the atmosphere or a vent line. When the cooling fluidic medium comes into contact with RF electrode 20 and evaporates, the resulting gas pressurizes the inside of electrode assembly 18. This can cause RF electrode 20 to partially inflate and bow out from front surface 26. The inflated RF electrode 20 can enhance the thermal contact with the skin and also result in some degree of conformance of RF electrode 20 to the skin surface. An electronic controller can be provided. The electronic controller sends a signal to open vent 38 when a programmed pressure has been reached.

Various leads 40 are coupled to RF electrode 20. One or more thermal sensors 42 are coupled to RF electrode. Suitable thermal sensors 42 include but are not limited to thermocouples, thermistors, infrared photo-emitters and a thermally sensitive diode. In one embodiment, a thermal sensor 42 is positioned at each corner of RF electrode 20. A sufficient number of thermal sensors 42 are provided in order to acquire sufficient thermal data of the skin surface. Thermal sensors 42 are electrically isolated from RF electrode 20.

Thermal sensors 42 measure temperature and can provide feedback for monitoring temperature of Rf electrode 20 and/or the tissue during treatment. Thermal sensors 42 can be thermistors, thermocouples, thermally sensitive diodes, capacitors, inductors or other devices for measuring temperature. Preferably, thermal sensors 42 provide electronic feedback to a microprocessor of the an RF generator coupled to RF electrode 20 in order to facilitate control of the treatment.

The measurements from thermal sensors 42 can be used to help control the rate of application of cooling fluidic medium. For example, the cooling control algorithm can be used to apply cooling fluidic medium to RF electrode 20 at a high flow rate until the temperature fell below a target temperature, and then slow down or stop. A PID, or proportional-integral-differential, algorithm can be used to precisely control RF electrode 20 temperature to a predetermined value.

Thermal sensors 42 can be positioned placed on back surface 24 of RF electrode 20 away from the tissue. This configuration is preferable ideal for controlling the temperature of the RF electrode 20. Alternatively, thermal sensors 42 can be positioned on front surface 26 of RF electrode 10 in direct contact with the tissue. This embodiment can be more suitable for monitoring tissue temperature. Algorithms are utilized with thermal sensors 42 to calculate a temperature profile of the treated tissue. Thermal sensors 42 can be used to develop a temperature profile of the skin which is then used for process control purposes to assure that the proper amounts of heating and cooling are delivered to achieve a desired elevated deep tissue temperature while maintaining skin tissue layers below a threshold temperature and avoid thermal injury. The physician can use the measured temperature profile to assure that he stays within the boundary of an ideal/average profile for a given type of treatment. Thermal sensors 42 can be used for additional purposes. When the temperature of thermal sensors 42 is monitored it is possible to detect when RF electrode 20 is in contact with the skin surface. This can be achieved by detecting a direct change in temperature when skin contact is made or examining the rate of change of temperature which is affected by contact with the skin. Similarly, if there is more than one thermal sensor 42, the thermal sensors 42 can be used to detect whether a portion of RF electrode 20 is lifted or out of contact with skin. This can be important because the current density (amperes per unit area) delivered to the skin can vary if the contact area changes. In particular, if part of the surface of RF electrode 20 is not in contact with the skin, the resulting current density is higher than expected.

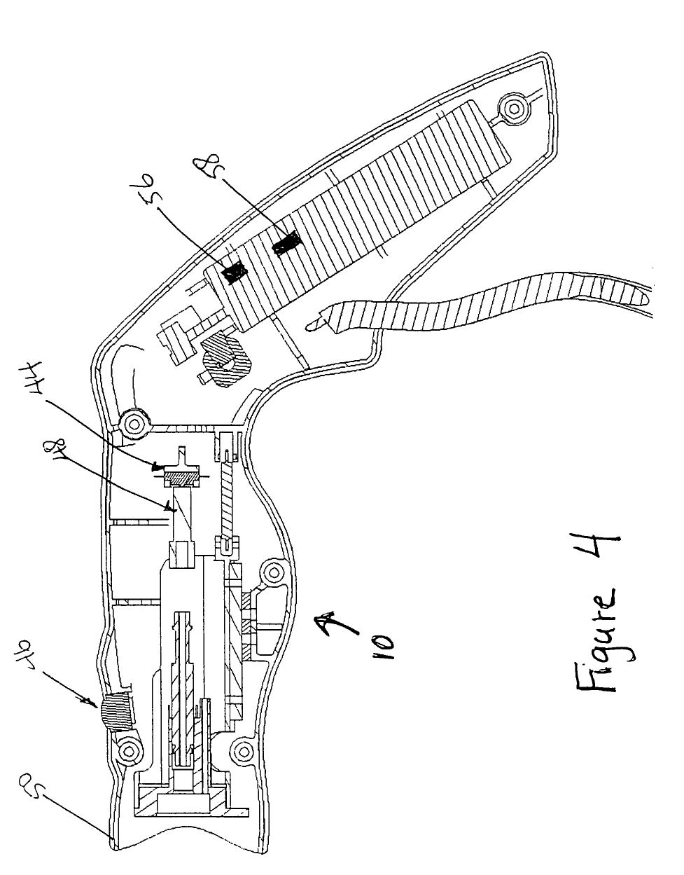

Referring now to FIG. 4, a force sensor 44 is also coupled to electrode assembly 18. Force sensor 44 detects an amount of force applied by electrode assembly 18, via the physician, against an applied skin surface. Force sensor 44 zeros out gravity effects of the weight of electrode assembly 18 in any orientation of front surface 26 of RF electrode 20 relative to a direction of gravity. Additionally, force sensor 44 provides an indication when RF electrode 20 is in contact with a skin surface. Force sensor 44 also provides a signal indicating that a force applied by RF electrode 20 to a contacted skin surface is, (i) below a minimum threshold or (ii) above a maximum threshold.

An activation button 46 is used in conjunction with the force sensor. Just prior to activating Rf electrode 20, the physician holds handpiece 10 in position just off the surface of the skin. The orientation of handpiece 10 can be any angle relative to the angle of gravity. To arm handpiece 10, the physician can press activation button 46 which tares force sensor 44, by setting it to read zero. This cancels the force due to gravity in that particular treatment orientation. This method allows consistent force application of RF electrode 20 to the skin surface regardless of the angle of handpiece 10 relative to the direction of gravity.

RF electrode 20 can be a flex circuit, which can include trace components. Additionally, thermal sensor 42 and force sensor 44 can be part of the flex circuit. Further, the flex circuit can include a dielectric that forms a part of RF electrode 20.

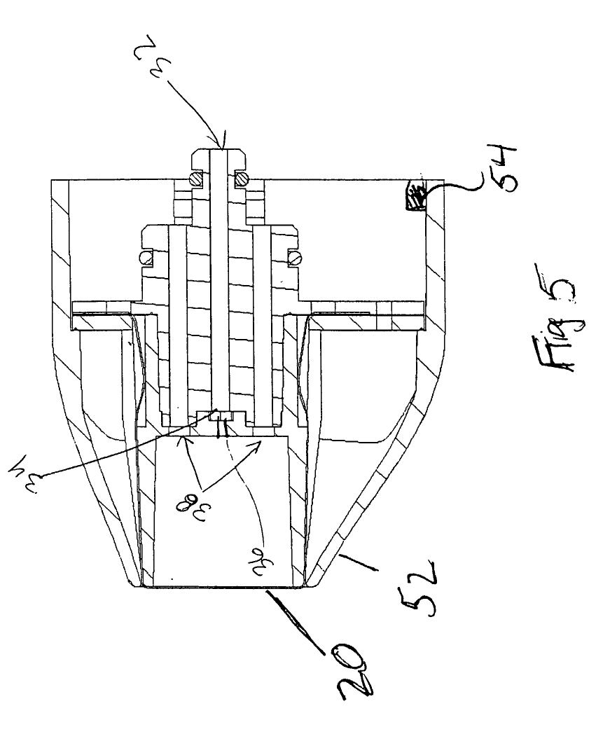

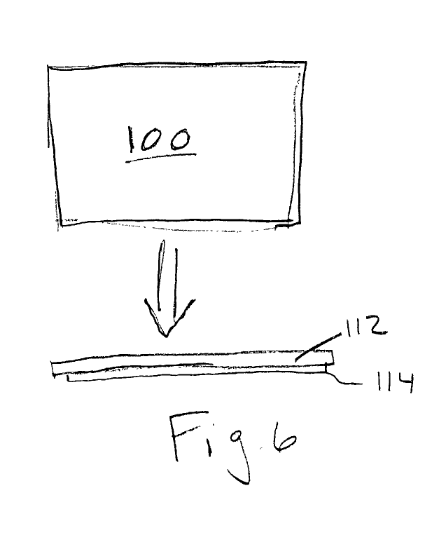

Electrode assembly 18 can be moveable positioned within handpiece housing 12. In one embodiment, electrode assembly 18 is slideably moveable along a longitudinal axis of handpiece housing 12. Electrode assembly 18 can be rotatably mounted in handpiece housing 12. Additionally, RF electrode 20 can be rotatably positioned in electrode assembly 18. Electrode assembly 18 can be removably coupled to handpiece housing 12 as a disposable or non-disposable insert 52, see FIG. 5. For purposes of this disclosure, electrode assembly 18 is the same as insert 52. Once movably mounted to handpiece housing 12, insert 52 can be coupled to handpiece housing 12 via force sensor 44. Force sensor 44 can be of the type that is capable of measuring both compressive and tensile forces. In other embodiments, force sensor 44 only measures compressive forces, or only measures tensile forces.

Insert 52 can be spring-loaded with a spring 48. In one embodiment, spring 48 biases RF electrode 20 in a direction toward handpiece housing 12. This pre-loads force sensor 44 and keeps insert 52 pressed against force sensor 44. The pre-load force is tared when activation button 46 is pressed just prior to application of RF electrode 20 to the skin surface.

A shroud 50 is optionally coupled to handpiece 10. Shroud 50 serves to keep the user from touching insert 52 during use which can cause erroneous force readings.

A non-volatile memory 54 can be included with insert 52. Additionally, non-volatile memory can be included with handpiece housing 12. Non-volatile memory 54 can be an EPROM and the like. Additionally, a second non-volatile memory 56 can be included in handpiece housing 12 for purposes of storing handpiece 10 information such as but not limited to, handpiece model number or version, handpiece software version, number of RF applications that handpiece 10 has delivered, expiration date and manufacture date. Handpiece housing 12 can also contain a microprocessor 58 for purposes of acquiring and analyzing data from various sensors on handpiece housing 12 or insert 52 including but not limited to thermal sensors 42, force sensors 44, fluid pressure gauges, switches, buttons and the like. Microprocessor 58 can also control components on handpiece 10 including but not limited to lights, LEDs, valves, pumps or other electronic components. Microprocessor 58 can also communicate data to a microprocessor of the RF generator.

Non-volatile memory 54 can store a variety of data that can facilitate control and operation of handpiece 10 and its associated system including but not limited to, (i) controlling the amount of current delivered by RF electrode 20, (ii) controlling the duty cycle of the fluid delivery member 22, (iii) controlling the energy delivery duration time of the RF electrode 20, (iv) controlling the temperature of RF electrode 20 relative to a target temperature, (v) providing a maximum number of firings of RF electrode 20, (vi) providing a maximum allowed voltage that is deliverable by RF electrode 20, (vii) providing a history of RF electrode 20 use, (viii) providing a controllable duty cycle to fluid delivery member 22 for the delivery of the cooling fluidic medium to back surface 24 of RF electrode 20, (ix) providing a controllable delivery rate of cooling fluidic medium delivered from fluid delivery member 22 to back surface 24, and the like.

Handpiece 10 can be used to deliver thermal energy to modify tissue including, but not limited to, collagen containing tissue, in the epidermal, dermal and subcutaneous tissue layers, including adipose tissue. The modification of the tissue includes modifying a physical feature of the tissue, a structure of the tissue or a physical property of the tissue. The modification can be achieved by delivering sufficient energy to cause collagen shrinkage, and/or a wound healing response including the deposition of new or nascent collagen.

Handpiece 10 can be utilized for performing a number of treatments of the skin and underlying tissue including but not limited to, (i) dermal remodeling and tightening, (ii) wrinkle reduction, (iii) elastosis reduction, (iv) sebaceous gland removal/deactivation, (v) hair follicle removal, (vi) adipose tissue remodeling/removal, (vii) spider vein removal, and the like.

In various embodiments, handpiece 10 can be utilized in a variety of treatment processes, including but not limited to, (i) pre-cooling, before the delivery of energy to the tissue has begun, (ii) an on phase or energy delivery phase in conjunction with cooling and (iii) post cooling after the delivery of energy to tissue has stopped.

Handpiece 10 can be used to pre-cool the surface layers of the target tissue so that when RF electrode 20 is in contact with the tissue, or prior to turning on the RF energy source, the superficial layers of the target tissue are already cooled. When RF energy source is turned on or delivery of RF to the tissue otherwise begins, resulting in heating of the tissues, the tissue that has been cooled is protected from thermal effects including thermal damage. The tissue that has not been cooled will warm up to therapeutic temperatures resulting in the desired therapeutic effect.

Pre-cooling gives time for the thermal effects of cooling to propagate down into the tissue. More specifically, pre-cooling allows the achievement of a desired tissue depth thermal profile, with a minimum desired temperature being achieved at a selectable depth. The amount or duration of pre-cooling can be used to select the depth of the protected zone of untreated tissue. Longer durations of pre-cooling produce a deeper protected zone and hence a deeper level in tissue for the start of the treatment zone. The opposite is true for shorter periods of pre-cooling. The temperature of front surface 26 of RF electrode 20 also affects the temperature profile. The colder the temperature of front surface 26, the faster and deeper the cooling, and vice verse.

Post-cooling can be important because it prevents and/or reduces heat delivered to the deeper layers from conducting upward and heating the more superficial layers possibly to therapeutic or damaging temperature range even though external energy delivery to the tissue has ceased. In order to prevent this and related thermal phenomena, it can be desirable to maintain cooling of the treatment surface for a period of time after application of the RF energy has ceased. In various embodiments, varying amounts of post cooling can be combined with real-time cooling and/or pre-cooling.

In various embodiments, handpiece 10 can be used in a varied number of pulse on-off type cooling sequences and algorithms may be employed. In one embodiment, the treatment algorithm provides for pre-cooling of the tissue by starting a spray of cryogenic cooling fluidic medium, followed by a short pulse of RF energy into the tissue. In this embodiment, the spray of cryogenic cooling fluidic medium continues while the RF energy is delivered, and is stopping shortly thereafter, e.g. on the order of milliseconds. This or another treatment sequence can be repeated again. Thus in various embodiments, the treatment sequence can include a pulsed sequence of cooling on, heat, cooling off, cooling on, heat, cool off, and with cooling and heating durations on orders of tens of milliseconds. In these embodiments, every time the surface of the tissue of the skin is cooled, heat is removed from the skin surface. Cryogenic cooling fluidic medium spray duration, and intervals between sprays, can be in the tens of milliseconds ranges, which allows surface cooling while still delivering the desired thermal effect into the deeper target tissue.

In various embodiments, the target tissue zone for therapy, also called therapeutic zone or thermal effect zone, can be at a tissue depth from approximately 100 μm beneath the surface of the skin down to as deep as 10 millimeters, depending upon the type of treatment. For treatments involving collagen contraction, it can be desirable to cool both the epidermis and the superficial layers of the dermis of the skin that lies beneath the epidermis, to a cooled depth range between 100 μm two millimeters. Different treatment algorithms can incorporate different amounts of pre-cooling, heating and post cooling phases in order to produce a desired tissue effect at a desired depth.

Various duty cycles, on and off times, of cooling and heating are utilized depending on the type of treatment. The cooling and heating duty cycles can be controlled and dynamically varied by an electronic control system known in the art. Specifically the control system can be used to control cooling fluidic medium valve member 16 and the RF power source.

In another embodiment of the present invention, all or a portion of the skin surface to be treated is marked before or after electromagnetic energy is delivery to and through the skin surface. The marking indicates to an operator those portions of the skin surface that will be, or have been exposed to electromagnetic energy.

The marking can be achieved with the use of a colorant that is on a substrate and subsequently transferred from the substrate to the skin surface. Colorant can be in the form of a dry solid on the substrate. Suitable substrates include but are not limited to paper, plastic, fabric, and the like. To transfer the colorant to the skin, the skin can be wetted and then the substrate is applied directly, with or without much pressure, to a selected skin surface to be treated. Thereafter, the substrate is peeled away and removed from the skin surface and at least a portion of the colorant previously on the substrate remains on the skin surface. In this embodiment, an adhesive and a membrane are not utilized. A membrane can be employed if it does not interfere with the controlled delivery of electromagnetic energy to a desired tissue site. For example, a membrane can be employed with the colorant with a variety of different energy sources. The membrane can be transparent to the delivery of the electromagnetic energy to the tissue site.

The marking can be in the form of a pattern including but not limited to a grid pattern of different geometries. These patterns are then utilized to assist in the delivery of electromagnetic energy to the tissue site. The pattern can be visible prior to the delivery of energy or become visible after electromagnetic energy is delivered. Additionally, the pattern can indicate where to deliver electromagnetic energy and where not to treat. In one embodiment, electromagnetic energy is delivered to only a portion of the patterned areas, leaving other areas non-treated. The pattern can be in a shape to substantially match the shape of the energy delivery device, e.g., electrode, or other pattern which aids the user in targeting successive treatment areas. The pattern can subsequently be removed from the skin surface by a variety of different methods, including but not limited to, being wiped off or removed from the skin surface on which it has been applied by washing with water, soap, alcohol and other removal compositions, and the like.

The pattern can be attached to the skin surface as part of a layered applique. In one embodiment, the image of the pattern is created on a computer, printed with a printer attached to the computer, incorporated in an image-bearing laminate, and then applied to the skin surface.

In one embodiment, the pattern, made from one or more dyes, colorant, ink, thermo or photo-sensitive material and the like (hereafter collectively “colorant”) is removable after the procedure is completed. A transfer sheet can be utilized. In a specific embodiment, a transfer sheet is provided that includes a substrate and a pattern layer on at least one surface of the substrate. The pattern layer of the transfer sheet is then wetted with a transfer solution containing, by way of illustration and without limitation, lower alcohols. A transfer sheet is then brought into contact with a selected patient skin surface onto which the pattern is to be transferred in such a manner that the pattern layer contacts the skin surface. The transfer sheet is maintained in contact with the receiving surface under pressure. The transfer sheet is then peeled from the receiving surface to leave the transferred pattern on the skin surface. The pattern can be on a water-soakable release-paper substrate that is coated with the colorant along with an optional protective layer including but not limited to, polyvinylbutyral and the like.

The pattern can be conformable to the skin surface in order to be positioned on a variety of different skin surfaces and countours. Colorant is preferably a hypo-allergenic material which can be smoothly applied over the desired area of the patient's skin and which is sufficiently flexible and strong enough to maintain the pattern whether applied to a relatively flat and smooth part of the body, such as the patient's back, or whether applied to a curved part of the body, such as the patient's arm or shoulder. The preferred materials minimize bleeding or spreading of the colorant beyond the pattern. Optionally, included is an adhesive that can be hypo-allergenic, pressure-sensitive. The adhesive can also be water resistant and have properties that enhance adherence to the skin surface. Suitable adhesives include acrylates, silicones and synthetic rubbers, although many other types of adhesives can be used.

The exact formulation of the adhesive depends on several factors including how long it is intended to be adhered to the patient's skin, and the area of the body to which the pattern is applied and the like. If the adhesive is overly aggressive or tacky for its intended purpose, it can be made less aggressive or less tacky by adding glycerides. The adhesive and colorant are removable without causing undue pain or causing removal of layers of skin. In one embodiment, the adhesive and the colorant are the same. The colorant can penetrate the skin or not significantly penetrate the skin.

Examples of suitable colorants include but are not limited to henna dye, disperse dyes, oil dyes, nitro dyes, such as 2,4-diamino anisole, base dyes and acid dyes, Rhodamine B Stearate (Red 215), Tetrachlorotetrabromofluorescein (Red 218), Tetrabromofluorescein (Red 223), Medical Scarlet (Red 501), Sudan Red III (Red 225), Oil Red XO (Red 505), FD & C Red No. 4, Disperse Red Orange Dyes, Dibromofluorescein (Orange 201), Diiodofluorescein (Orange 206), Orange SS (Orange 403), D & C Orange No. 4, Yellow Dye, Fluorescein (Yellow 201), Quinoline Yellow SS (Yellow 204), Yellow AB (Yellow 404), Yellow OB (Yellow 405), FD & C Yellow No. 6, D & C Yellow No. 10, Green Dyes, Quinazarin Green SS (Green 202), D & C Green No. 5, Blue and Purple Dyes, Indigo, Sudan Blue B (Blue 403), Arizroll Purple (Violet 201), D & C Violet No. 2, Disperse Blue, Disperse Violet, FD & C Blue No. 1, Brown and Black Dyes, and the like.

The pattern can be applied to the patient's skin surface as solutions or suspensions (dispersions) with the fluid phase being a liquid or a gel. The fluid phase from which the colorant is delivered can be aqueous or nonaqueous. The aqueous fluid phase may be essentially completely water but can also include cosolvents, permeation enhancers, adjuvants, agents which alter or enhance the hydrophobic or hydrophillic character of the skin, and the like.

These can include organic liquids such as alcohols, ketones, halohydrocarbons, ethers and the like. Examples of suitable alcohols include benzyl alcohol, methanol, ethanol, isopropanol, n-butanol and cyclohexanol and the like. Ketones can be used are non-irritating to the patient's skin. Examples of ketones include dimetylketone, methyl-ethyl ketone and the like. Halohydrocarbon liquids include chlorohydrocarbons with 1 to 3 carbon atoms and the like. Known skin permeation enhancers include DMSO and the like.

The colorant solutions, suspensions or gels can include cationic, anionic or nonionic surfactants such as the SPANs or the TWEENs. One can also include materials to alter or stabilize the dye's pH. This pH will be based to a great extent upon the colorants employed but can be selected to not be irritating or disruptive to the patient's skin. Acid pH's down to about 3 are typically not damaging. Further, pH's of up to about 11 can be employed with minimal skin saponification. Simple inorganic acids and bases can be used to set the pH such as the mineral acids and the alkali metal and the alkaline earth metal hydroxides and carbonates and the like. Good results can be achieved using organic acids such as acetic acid, citric acid, the benzene sulfonic acids, naphthalene sulfonic acid. These weaker acids, as well as the phosphorous-based acids can buffer systems which can be selected to advantageously buffer the pH into the desired pH's wherever in the 2 to 11 range.

These colorant solutions may be employed as gels as well as liquids or suspensions. Gels can be formed using the liquid substrates together with synthetic thickeners or gelling agents such as polyvinyl alcohol, polyglycols, and the like. Naturally occurring or modified naturally occurring gelling agents may also be employed, which include, for example, gelatine, starch and other oligosaccharides including the gums such as xanthene gum, gum arabic, gum guar, and the like, all of which are well known in the art as safe and useful thickeners and gelling agents.

As noted above, a variety of other types of colorants, both naturally occurring and synthetic, may be used. Further, the colorant may be in liquid, gel or solid form.

In one embodiment, the printable material on which the pattern is printed is a coated release sheet with a backing sheet and a printable releasable coating. When the backing sheet is wetted, the coating separates from the backing sheet. The pattern is printed on the coating, so that the pattern is separable from the backing sheet. The coating is waterproof so that it protects the pattern from possible damage when the backing sheet is wetted. The release coating also winds up being the outer layer of the pattern-bearing laminate that forms the pattern, protecting the pattern from damage by washing, rubbing and chaffing until it is desired to remove the pattern.

In one embodiment, the pattern and coating are applied with a double-sided medical film, so that the film is between a patient's skin and the pattern, and attached to both with adhesive. The pattern is layered between the film and the coating of the release sheet. The film both protects the patient from any harmful inks that may have been used in printing the pattern, and makes the pattern last longer once applied by reinforcing the pattern and coating. It has the added advantage of being releasable from the skin when peeled back, so that the pattern may be removed from the skin at any time.

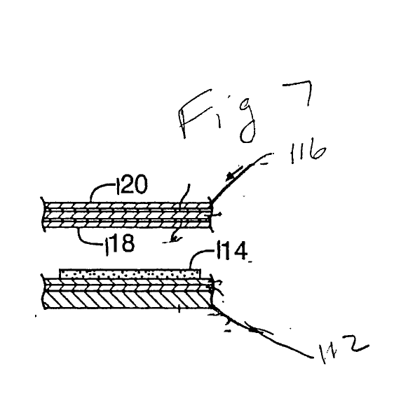

Referring now to FIG. 6, one embodiment for creating the pattern is by use of a device 100 that transfers colorant to a substrate including but not limited to an inkjet printer. Other techniques of printing the pattern on a coated release sheet 112 may be used, including photocopiers, commercial printers, heat transfers, and hand drawings and the like. A printed pattern 114 is on coated release sheet 112.

As illustrated in FIG. 7, printed pattern 114 and coated release sheet 112 are shown in proximity to a film 116. Film 116 can be a double-sided adhesive tape, typically with a protective backing 118 on one side and a protective backing 120 on the other side. One of protective backings 118 and 120 is removed, and film 116 is attached to pattern 114 and release sheet 112.

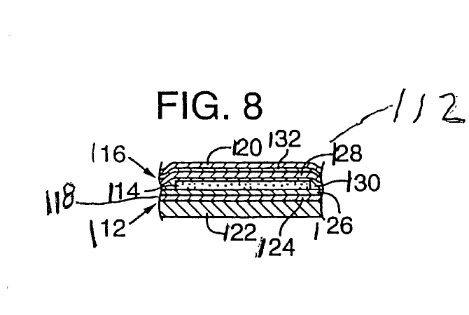

Turning now to FIG. 8, one embodiment of the specific components of coated release sheet 112 and film 116 are shown. In this embodiment, coated release sheet 112 includes a backing sheet 122 and a releasable, printable coating, preferably including a waterproof release layer 124, and a printable layer 126. The particular choice of waterproof release layer 124 and printable layer 126 depends on the type of release mechanism used, and the types of inks used. Furthermore, a single-layer coating may be used.

Preferably, release layer 124 is waterproof, so that the releasable coating 124 protects pattern 114 from damage when backing sheet 122 is released. A waterproof coating can be included to protect pattern 114 from other damage. A suitable coated release sheet is available from Arkwright, of Rhode Island, under their product designation L291-20A.

Film 16 can be relatively thin, flexible and clear. For example, a double-coated medical film available from 3M Medical Specialties, 3M Health Care Product No. 1512 is suitable. This medical film includes a transparent polyethylene layer 128, having a thickness of 1.5-mils. Polyethylene is sufficiently impenetrable to the ink used to create pattern 114 to protect a the patient from most any potential harmful components of the ink.

An adhesive 130 is on a first face or side of film 128, and a similar adhesive 132 can be on a second side or face of film 128 opposite the first face. Preferably, adhesives 130 and 132 are hypoallergenic and can be, by way of illustration and without limitation an acrylate. Protective backings 118 and 120 can be bleached Kraft-glassine paper, silicone coated on both sides so that each releases easily from adhesives 130 and 132. The resulting thickness of film 116, excluding protective backings 118 and 120, can be approximately 3.4-mils. When first face of film 116 is attached to pattern 114 and release sheet 112 a sheet/pattern/film laminate 112/116 is created.

Film 116, protective backing 120 and adhesives 130 and 132 can be transparent, or at least translucent. After protective backing 20 i removed from film 116 adhesive 132 can then pressed into contact with a selected skin surface. Backing sheet 122 is then may be removed from laminate 112/116. For most release sheets, this can be accomplished by wetting backing sheet 122. The resulting pattern 114 is illustrated in FIG. 9 on a face skin surface. By removing protective backing 120 from laminate 112/116, an adhesive pattern 114 is obtained that is then applied to the skin surface. Backing sheet 122 then is released from the laminate 112/116, and pattern 114 remains on the skin surface encased between film 116 and the releasable coating of sheet 112.

### EXAMPLES

### Example I

Water-Soakable Release-Paper

SKINCAL (r)

Water-Insoluble Protective Coating

Solids 12 wt %

Polyvinylbutyral

Solvent (Volatiles) 88 wt %

Methanol

Ink jet Imaging Coating

Solids

Poly(2-Ethyl-2-Oxazoline) 14.94 wt %

Polymethyl Methacrylate Spheres 0.04 wt %

(particle size 10 microns)

Cellulose Acetate Propionate 1.60 wt %

Polyvinyl Pyrrolidone 1.25 wt %

Citric Acid 0.09 wt %

Solvent (Volatiles)

Methyl Ethyl Ketone 39.36 wt %

Propylene Glycol Mono Ethyl Ether 26.24 wt %

Ethanol 16.43 wt %

The gum-coated side of the SKINCAL(r) paper is treated with the above-described water-insoluble protective coating. The coating is applied using a No. 28 Mayer rod and dried for 3 minutes at 120 degree(s) F. Next, the above-described ink jet imaging coating is applied to the dried water-insoluble protective layer using a No. 40 Mayer rod and dried for 3 minutes at 120 degree(s) F. In this manner, an ink jet recording medium, suitable for forming the pattern is prepared.

### Example 2

Water Soakable Release Paper

SKINCAL (r)

Ink jet Imaging Coating layer

Solids

1. Copolymer of vinylpyrrolidone and dimethyl 50 wt. %

Ammonium methacrylate (ISP Technologies)

2. Copolymer of methyl methacrylate and 10 wt. %

hydroxyethyl methacrylate (Allied Colloids)

3. Methylated melamine-formaldehyde resin 10 wt. %

(Cytec Industries)

Solvent (Volatiles)

Methyl Ethyl Ketone 20 wt. %

The gum-coated side of the SKINCAL(r) paper is treated with the above-described highly cross-linked ink jet imaging coating. The coating is applied using a No. 40 Mayer rod and dried for 3 minutes at 120 degree(s) F. In this manner, an ink jet recording medium, suitable for forming the pattern is prepared.

The removable skin marking embodiments of the present invention can be used with a variety of different energy sources, including but not limited to RF, microwave, ultrasound, resistive heating, coherent and incoherent light, and the like, and for a variety of different devices for treating skin tissue. Examples of other medical devices include but are not limited to, lasers, microwave applicators, ultrasound applicators, electrosurgery devices and a host of other instruments. The marking devices and methods of the present invention can be used when there is a need for marking the skin prior to performing a procedure on it

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A method for creating a desired tissue effect, comprising:
marking a skin surface to create a marked skin surface;
providing a handpiece that includes a handpiece assembly coupled to an electrode assembly with at least one RF electrode that is capacitively coupled to a skin surface when at least a portion of the RF electrode is in contact with the skin surface; and
delivering RF energy from the RF electrode assembly to at least a portion of the marked skin surface.

2. The method of claim 1, wherein the skin surface is marked prior to a delivery of RF energy to the marked skin surface.

3. The method of claim 1, wherein a colorant supported on a substrate is used to create the marked skin surface.

4. The method of claim 3, wherein the substrate is removed from the marked skin surface prior to a delivery of RF energy to the marked skin surface.

5. The method of claim 1, wherein the marked skin surface is marked with a pattern.

6. The method of claim 5, wherein the pattern is removable.

7. The method of claim 5, wherein the pattern is a grid pattern.

8. The method of claim 1, further comprising: providing an atomizing delivery of a cooling fluidic medium to the RF electrode.

9. The method of claim 1, further comprising: delivering a controllable amount of a cooling fluidic medium to the RF electrode.

10. The method of claim 1, further comprising: delivering a cooling fluidic medium to a back surface of the RF electrode.

11. The method of claim 1, further comprising: evaporatively cooling the RF electrode and conductively cooling a skin surface in contact with the front side of the RF electrode.

12. The method of claim 1, wherein the fluid delivery member is configured to controllably deliver a cooling fluidic medium to a back surface of the RF electrode at substantially any orientation of the front surface of the RF electrode relative to a direction of gravity.

13. The method of claim 1, wherein the electrode assembly is sufficiently sealed to minimize flow of a cooling fluidic medium from a back surface of the RF electrode to a skin surface in contact with a front surface of the RF electrode.

14. The method of claim 1, wherein the RF electrode includes a conductive portion and a dielectric portion.

15. The method of claim 14, wherein the conductive portion includes metal.

16. A method for creating a tissue effect, comprising:
marking a skin epidermis surface;
providing an energy source;
creating a reverse thermal gradient through at least a portion of the skin epidermis surface where a temperature of the skin epidermis surface is lower than an underlying collagen containing tissue site; and
delivering energy from the energy source through the skin epidermis surface to the collagen containing tissue site for a sufficient time to induce collagen formation in the collagen containing tissue site while minimizing cellular necrosis of the skin epidermis surface to create a desired tissue effect.

17. The method of claim 16, wherein the skin epidermis surface is marked prior to a delivery of energy to the skin epidermis surface.

18. The method of claim 16, wherein a colorant supported on a substrate is used to mark the skin epidermis surface.

19. The method of claim 18, wherein the substrate is removed from the skin epidermis after colorant is delivered to the skin epidermis surface.

20. The method of claim 16, wherein formation of the collagen alters a consistency of the collagen containing tissue site.

21. The method of claim 16, wherein formation of the collagen changes the geometry of the collagen containing tissue site.

22. A method for creating a desired tissue effect, comprising:
marking a skin epidermis surface to create a marked skin epidermis surface;
providing an energy source;
cooling at least a portion of the marked skin epidermis surface;
delivering thermal energy to tissue underlying the at least a portion of the marked skin epidermis surface without creating substantial necrosis at the skin epidermis surface; and
creating a desired tissue effect.

23. The method of claim 22, wherein the skin epidermis surface is marked prior to a delivery of thermal energy to the skin epidermis surface.

24. The method of claim 22, wherein a colorant supported on a substrate is used to create the marked skin epidermis surface.

25. The method of claim 24, wherein the substrate is removed from the marked skin epidermis after colorant is delivered to the skin epidermis surface.

26. The method of claim 22, wherein the desired tissue effect is selected from skin remodeling, skin resurfacing, wrinkle removal, treatment of the sebaceous glands, treatment of hair follicles, treatment of adipose tissue, treatment of acne and treatment of spider veins.

27. A method for creating a desired tissue effect, comprising:
marking a skin epidermis surface to create a marked skin epidermis surface;
positioning an energy delivery surface of an electromagnetic delivery device on at least a portion of the marked skin epidermis surface;
creating a reverse thermal gradient on at least a portion of the marked skin epidermis surface, the reverse thermal gradient cooling the skin epidermis surface while heating underlying tissue, wherein a temperature of the marked epidermis skin surface is lower than a temperature of the underlying tissue;
contracting at least a portion of the underlying tissue while minimizing cellular destruction of the marked skin epidermis surface; and
creating a desired tissue effect.

28. The method of claim 27, wherein the skin epidermis surface is marked prior to a creating the reverse thermal gradient.

29. The method of claim 27, wherein a colorant supported on a substrate is used to create the marked skin epidermis surface.

30. The method of claim 29, wherein the substrate is removed from the marked skin epidermis after colorant is delivered to the skin epidermis surface.

31. The method of claim 27, wherein marked skin epidermis surface is a patterned marked epidermis skin surface.

32. The method of claim 27, wherein the marked skin epidermis surface provides guidance for delivery of electromagnetic energy to the skin epidermis surface.

33. A method for creating a tissue effect, comprising:
providing a substrate with a releasable colorant;
applying the substrate to a selected skin epidermis surface to mark a skin surface with the colorant;
providing an energy source;
creating a reverse thermal gradient through at least a portion of the skin epidermis surface where a temperature of the skin epidermis surface is lower than an underlying collagen containing tissue site; and
delivering energy from the energy source through the skin epidermis surface to the collagen containing tissue site for a sufficient time to induce collagen formation in the collagen containing tissue site while minimizing cellular necrosis of the skin epidermis surface to create a desired tissue effect.

34. The method of claim 33, wherein the skin epidermis surface is marked prior to a creating the reverse thermal gradient.

35. The method of claim 33, wherein the coating is a patterned coating.

36. The method of claim 33, wherein the coating is non-toxic to the skin epidermis.

37. The method of claim 36, wherein the coating is selected from the group henna, indigo, disperse dyes, oil dyes, nitro dyes, basic dyes and acid dyes.

38. The method of claim 33, wherein the coating is a dry coating.

39. The method of claim 33, wherein the coating is a gel coating.

40. The method of claim 33, wherein the coating is a liquid coating

41. A kit, comprising:
a substrate with a releasable colorant coating; and
a handpiece that includes a handpiece assembly coupled to an electrode assembly with at least one RF electrode that is capacitively coupled to a skin surface when at least a portion of the RF electrode is in contact with the skin surface.

42. A method for creating a tissue effect, comprising:
providing a substrate with a releasable coating;
releasing at least a portion of the releasable coating on a selected skin epidermis surface to create a marked skin epidermis surface;
using the marked skin epidermis surface to provide a guide for delivery of energy from an energy source to a tissue site through at least a portion of the marked skin epidermis surface.

43. The method of claim 42, wherein the skin epidermis surface is marked prior to a delivery of thermal energy to the skin epidermis surface.

44. The method of claim 42, wherein a colorant supported on a substrate is used to create the marked skin epidermis surface.

45. The method of claim 44, wherein the substrate is removed from the marked skin epidermis after colorant is delivered to the skin epidermis surface.

46. The method of claim 42, wherein the delivery of energy from the energy source to a tissue site through at least a portion of the marked skin epidermis surface creates a desired tissue effect.

47. The method of claim 46, wherein the desired tissue effect is selected from skin remodeling, skin resurfacing, wrinkle removal, treatment of the sebaceous glands, treatment of hair follicles, treatment of adipose tissue, treatment of acne and treatment of spider veins.

48. The method of claim 42, wherein the skin epidermis surface is marked prior to delivering the energy from the energy source to the tissue site through the at least a portion of the marked skin epidermis surface.

49. The method of claim 42, wherein the substrate is removed from the marked skin epidermis after at least a portion of the releasable coating is delivered to the skin epidermis surface.

50. The method of claim 42, wherein the marked skin epidermis surface is a patterned marked epidermis skin surface.

51. The method of claim 42, wherein the energy source is selected from RF, microwave, ultrasound, resistive heating, coherent and incoherent light.
